(19) United States
(12) Patent Application Publication
(10) Pub. No.: US 2013/0040313 A1
(43) Pub. Date: Feb. 14, 2013

(54) Nanofluidic biochemical sensors based on surface charge modulated ion current

(71) Applicants: Ali Afzali-Ardakani, Ossining, NY (US); Philip S. Waggoner, Fishkill, NY (US)

(72) Inventors: Ali Afzali-Ardakani, Ossining, NY (US); Philip S. Waggoner, Fishkill, NY (US)

(73) Assignee: INTERNATIONAL BUSINESS MACHINES CORPORATION, Armonk, NY (US)

(21) Appl. No.: 13/206,588

(22) Filed: Aug. 10, 2011

(51) Int. Cl. G01N 33/543; G01N 30/96; C12M 1/34

(52) U.S. Cl. 435/7.8; 436/501; 435/7.94; 435/287.1; 422/69

(57) ABSTRACT

Biological and chemical sensors based on surface charge changes in a pore or channel, such as a nanopore or nanochannel, are employed to detect targeted analytes in an electrolyte solution having a low ion concentration. Receptors within the pore or channel capture a targeted analyte, causing a change in surface charge that affects ionic conductance. The change in ionic conductance is detected, evidencing the presence of the targeted analyte. A secondary tag may be introduced to the pore or channel for binding with a captured analyte in certain circumstances for causing a change in the surface charge.

### FIELD OF THE INVENTION

The present invention relates to the physical arts and, more particularly, to nanofluidic and microfluidic sensors and the like.

### BACKGROUND OF THE INVENTION

Nanoscale fluidic devices include pores and/or channels formed in selected substrates. A solid-state nanopore may be fabricated through TEM (transmission electron microscope) drilling through a selected substrate. Solid-state nanopores can be used to analyze biological proteins.

Nanofluidic channels may be fabricated by serial electron beam lithography in order to reach the desired dimensions. Channels can also be fabricated using photolithography, nanoimprint lithography and nanotransfer lithography.

Nanopores have been used as sensors for molecules such as DNA. A small passage may be arranged to separate two electrolyte-filled reservoirs, at least one of which contains target molecules. The target molecules can be drawn through the passage and their presence detected as a current drop. Using high ion concentration, the pore functions as an electrical resistor wherein the resistance scales as length over cross-sectional area. Changes in the pore cross-sectional area may occur when floppy and somewhat coiled single stranded DNA hybridizes with its complementary strand. Double stranded DNA can be fairly rigid and rod-like. The pore diameter accordingly decreases substantially resulting in a physical blockage of the ion current through the pore. The change in current can be detected.

### SUMMARY OF THE INVENTION

Principles of the invention provide techniques for the detection of analytes using microfluidic and nanofluidic sensors. In one aspect, an exemplary method includes the step of obtaining a device comprising a fluidic passage including a receptor layer for capturing a selected analyte, the fluidic passage including the receptor layer having at least one dimension of one thousand nanometers or less. An electrolyte solution containing one or more molecules of the selected analyte flows through the fluidic passage such that the selected analyte is captured by the receptor layer. The capture of the analyte causes a change in surface charge on the receptor layer. The electrolyte solution used in the method has a sufficiently low salt concentration that the surface charge causes a material effect on ionic conductance through the fluidic passage. The ionic conductance through the fluidic passage is detected. Changes in conductance reflect the capture of the targeted analyte.

In another aspect, an exemplary method comprises flowing an electrolyte solution through a fluidic passage. The passage includes a receptor layer for capturing a selected analyte and causing a change in surface charge within the fluidic passage upon capturing the selected analyte. The fluidic passage including the receptor layer has at least one dimension of one thousand nanometers or less. The electrolyte solution has a sufficiently low salt concentration that surface charge within the fluidic passage causes a material effect on ionic conductance through the fluidic passage. The exemplary method further includes detecting the ionic conductance through the fluidic passage.

A further exemplary method involves the use of a secondary tag capable of binding to a targeted analyte. The method comprises flowing an electrolyte solution through a fluidic passage including a receptor layer for capturing a selected analyte, the fluidic passage including the receptor layer having at least one dimension of one thousand nanometers or less. The electrolyte solution has a sufficiently low salt concentration that surface charge within the fluidic passage can cause a material effect on ionic conductance through the fluidic passage. The method further includes introducing a secondary tag capable of binding with the selected analyte into the fluidic passage and providing a surface charge within the fluidic passage upon binding with the selected analyte, and detecting the ionic conductance through the fluidic passage.

An exemplary system in accordance with the invention comprises a substrate including a fluidic passage having a surface including a receptor layer for capturing an analyte and causing a change in surface charge upon capturing the analyte. The fluidic passage including the receptor layer has at least one dimension of one thousand nanometers or less. A first fluidic chamber and a second fluidic chamber are in fluid communication with the fluidic passage. The system includes a voltage source for applying a voltage across the fluidic passage and a detecting device for detecting changes in electrical conductance through the fluidic passage. An electrolyte solution in the first fluidic chamber has a sufficiently low salt concentration that a change in the surface charge resulting from capture of the analyte by the receptor layer when the electrolyte solution flows through the fluidic passage causes a material effect in ionic conductance through the fluidic passage.

As used herein, “facilitating” an action includes performing the action, making the action easier, helping to carry the action out, or causing the action to be performed. Thus, by way of example and not limitation, instructions executing on one processor might facilitate an action carried out by instructions executing on a remote processor, by sending appropriate data or commands to cause or aid the action to be performed. For the avoidance of doubt, where an actor facilitates an action by other than performing the action, the action is nevertheless performed by some entity or combination of entities.

One or more embodiments of the invention or elements thereof can be implemented in the form of a computer program product including a computer readable storage medium with computer usable program code for performing the method steps indicated. Furthermore, one or more embodiments of the invention or elements thereof can be implemented in the form of a system (or apparatus) including a memory, and at least one processor that is coupled to the memory and operative to perform exemplary method steps. Yet further, in another aspect, one or more embodiments of the invention or elements thereof can be implemented in the form of means for carrying out one or more of the method steps described herein; the means can include (i) hardware module(s), (ii) software module(s) stored in a computer readable storage medium (or multiple such media) and implemented on a hardware processor, or (iii) a combination of (i) and (ii); any of (i)-(iii) implement the specific techniques set forth herein.

Techniques of the present invention can provide substantial beneficial technical effects. For example, one or more embodiments may provide one or more of the following advantages:
Allows point-of-care diagnostics/biosensors;
High sensitivity applications and trace detection possible;
Minimal equipment requirements;
Detection of small, charged analytes.

These and other features and advantages of the present invention will become apparent from the following detailed description of illustrative embodiments thereof, which is to be read in connection with the accompanying drawings.

### BRIEF DESCRIPTION OF THE DRAWINGS

FIG. 1A is a graph illustrating electrical conductance as a function of electrolyte concentration for nanopores formed in silicon nitride shown in FIGS. 1B, 1C and 1D;

FIG. 2A is a graph illustrating electrical conductance as a function of electrolyte concentration for nanopores formed in titanium nitride shown in FIGS. 2B, 2C and 2D;

FIG. 3 is a graph illustrating electrical conductance as a function of electrolyte concentration for a nanopore in titanium oxide prior to and following formation of an oxide layer;

FIG. 4 is schematic illustration showing surface charge density in a channel and its effect on ions in an electrolyte solution;

FIG. 5 is a graph showing conductance in nanosiemens as a function of KCl concentration;

FIG. 6 is a schematic illustration of a pore including a receptor layer;

FIG. 7 is a schematic illustration of the pore shown in FIG. 6 including analytes captured by the receptor layer;

FIG. 8 is a schematic illustration of a device including a nanopore including a receptor layer;

FIGS. 9A and 9B show the capture of a D-glucose analyte by a boronic acid receptor layer;

FIG. 10 is a graph showing a change in ionic conductance in an electrolyte depending on ion concentration;

FIG. 11 is a graph showing conductance in picosiemens of a nanopore following introduction of glucose and subsequent flushing with glucose-free electrolyte solution;

FIG. 12 is a schematic illustration of an assembly for measuring conductance in a fluidic channel having a surface including a receptor layer;

FIG. 13 is a graph illustrating current through the fluidic channel of FIG. 12 as a function of time;

FIG. 14 is a flow diagram illustrating the processing of a sample containing an analyte;

FIG. 15 includes a graph illustrating current through a single pore during the capture of analyte molecules within the pore;

FIG. 16 includes a graph illustrating current through an array of pores during the capture of analyte molecules within the array of pores;

FIG. 17 shows a system for detecting a plurality of different analytes that may be present in a test sample, and

FIG. 18 depicts a computer system that may be useful in implementing one or more aspects and/or elements of the invention.

### DETAILED DESCRIPTION OF PREFERRED EMBODIMENTS

The detection of biological molecules such as proteins, DNA, and enzymes can be useful in the field of diagnostics. The present invention provides techniques employing passages such as microfluidic and/or nanofluidic pores or channels to detect such molecules. Changes in ionic conductance can be detected resulting from surface charge changes of the passage. The binding of selected molecules to the surface of the passage can allow the detection of the selected molecules as discussed further below. Sensing devices capable of using such techniques are further provided by the invention for detecting selected molecules.

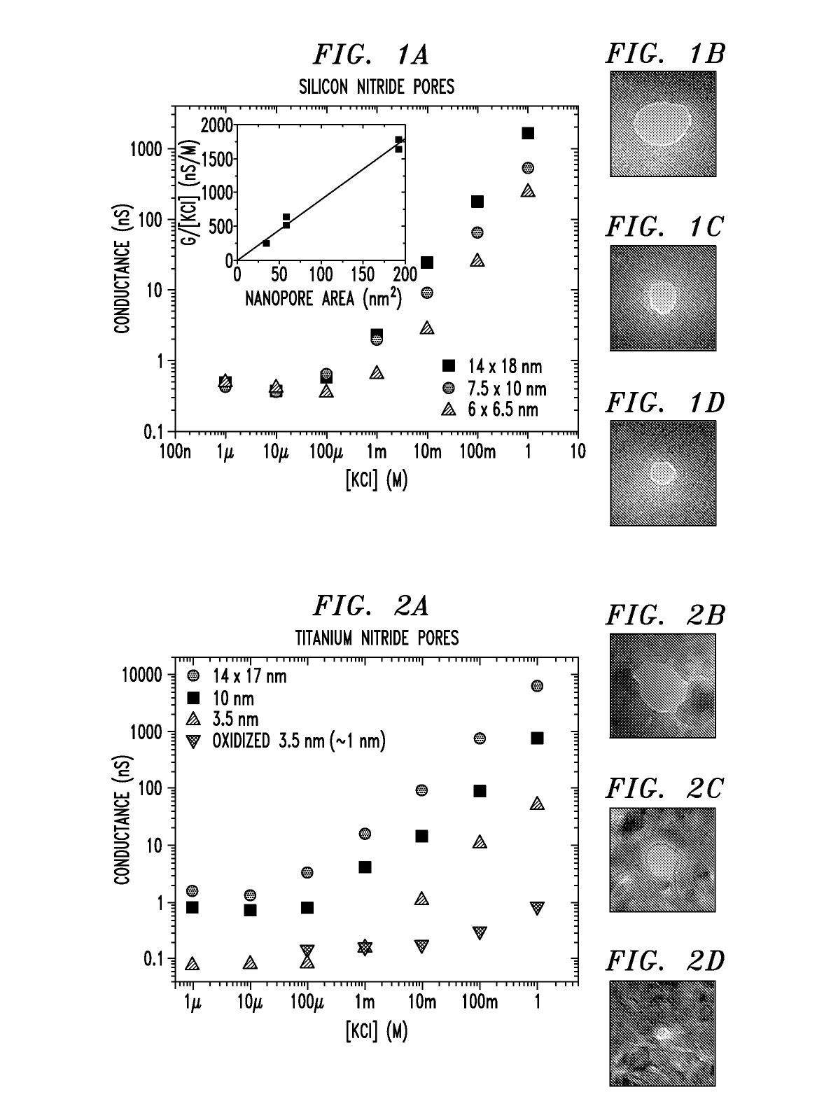

Nanopore and nanochannel ion conductance at high ion concentrations is dictated by pore geometry and ion concentration. At low concentrations however, surface charge substantially controls the number of ions in the pore or channel and thus its conductance. Referring to FIGS. 1(A)-(D) and 2(A)-(D), the conductance through nanopores of various sizes formed in silicon nitride and titanium nitride, respectively, is shown. The conductance tends to increase with an increase in the ion concentration of the KCl electrolyte solution as well as an increase in pore size.

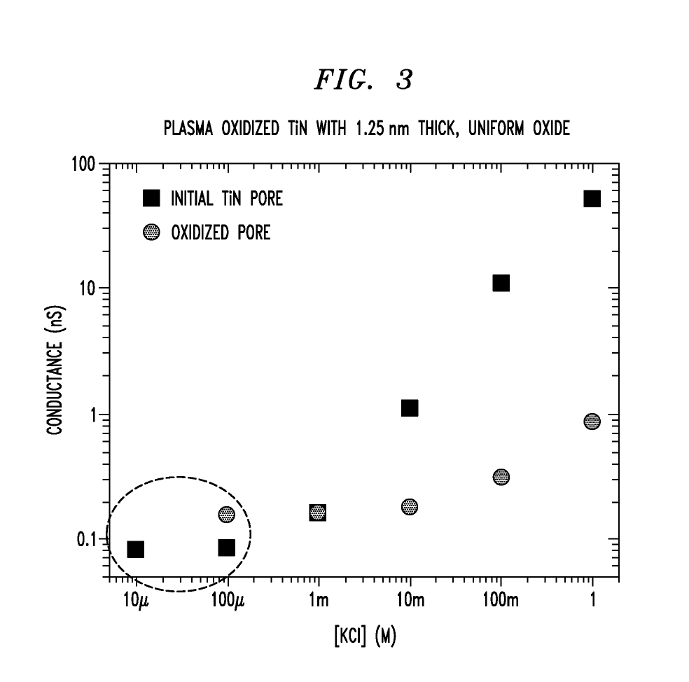

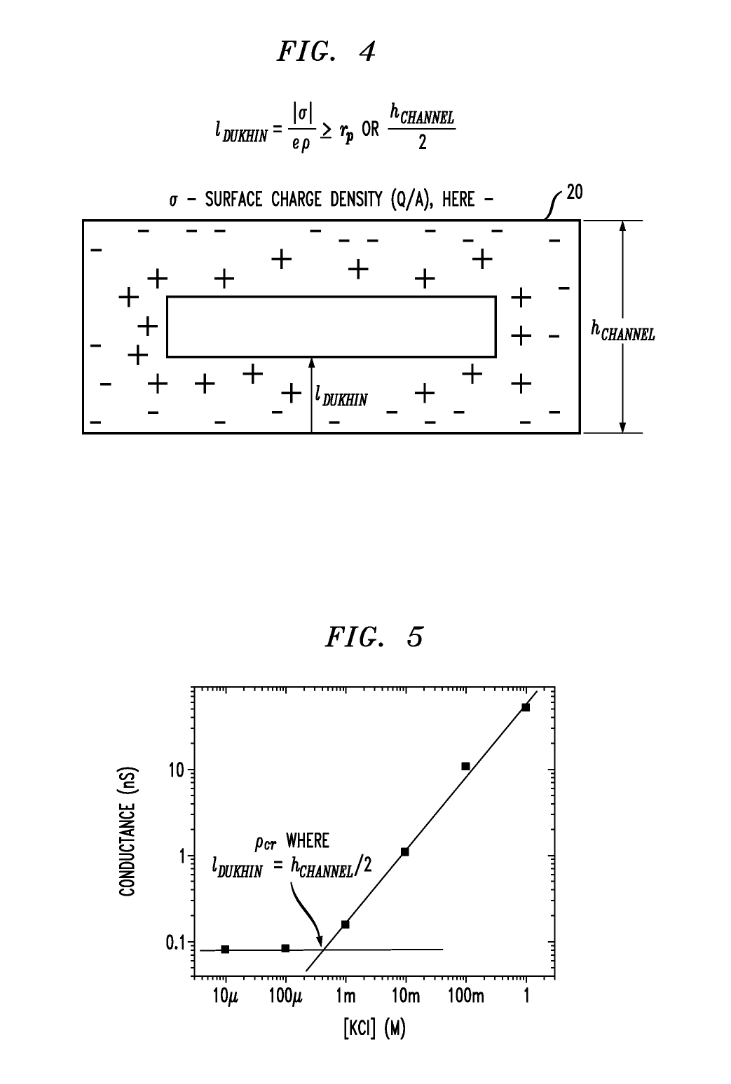

Referring to FIG. 3, it can be observed that, at low ion concentrations, the conductance through a smaller nanopore can be greater than through a nanopore of larger size under certain conditions. In this exemplary test, the conductance is greater through a plasma oxidized TiN nanopore having a 1.25 nm thick, uniform oxide than it is through a 3.5 nm TiN nanopore having no oxide coating. At relatively high KCl concentrations, conductivity of the oxidized nanopore is reduced due to the reduced nanopore diameter. However, at low concentrations, conductivity is higher because of the higher surface charge of the oxidized nanopore surface. FIG. 4 schematically illustrates the effect that surface charge can have in a rectangular passage 20 of height “h”. Ions in solution are influenced by the electric fields on the surface of a pore or channel out to the “Dukhin length.” The negative charges shown in FIG. 4 represent surface charges while the positive signs represent positive ions within a Dukhin length of the surface that counteract the surface charge. Ion conductance through the pore/channel is saturated at a constant value if 1_{Dukhin}=σ/eρ≧rₚ (pore radius) or h_{channel}/2 where ρ is counter-ion concentration and σ is surface charge density. Gₛₐₜ=2μw|σ|/l, where w and l are the width and length of the passage, respectively. At saturation, the counter-ion number in the passage is constant leading to constant conductance as shown in FIG. 5.

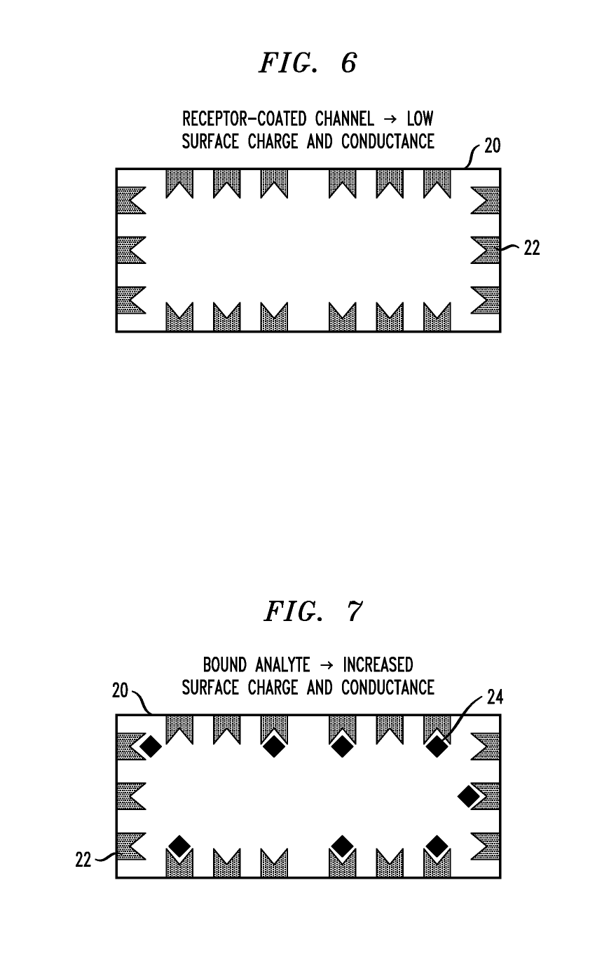

FIGS. 6 and 7 show the passage 20 including a receptor layer 22. FIG. 7 further shows analyte molecules 24 captured by the receptor layer 22. The receptor layer 22 can be comprised of receptor molecules that target particular analyte(s). Non-limiting examples include chemical receptors, antibodies, and oligonucleotides. Chelating molecules such a tri- or bi-pyridine that are known to capture multivalent, heavy metal ions in water may also possibly be employed. In one exemplary testing process, the ionic conductance through the passage (channel or pore) can be measured using calibration fluid, preferably an electrolyte solution having a low salt concentration such as 0.1 mM KCl. The solution containing (or possibly containing) the analyte of interest is introduced while ionic conductance continues to be measured. The passage is then flushed with calibration fluid and the new ionic conductance is measured. Real-time sensing of a targeted analyte can be provided.

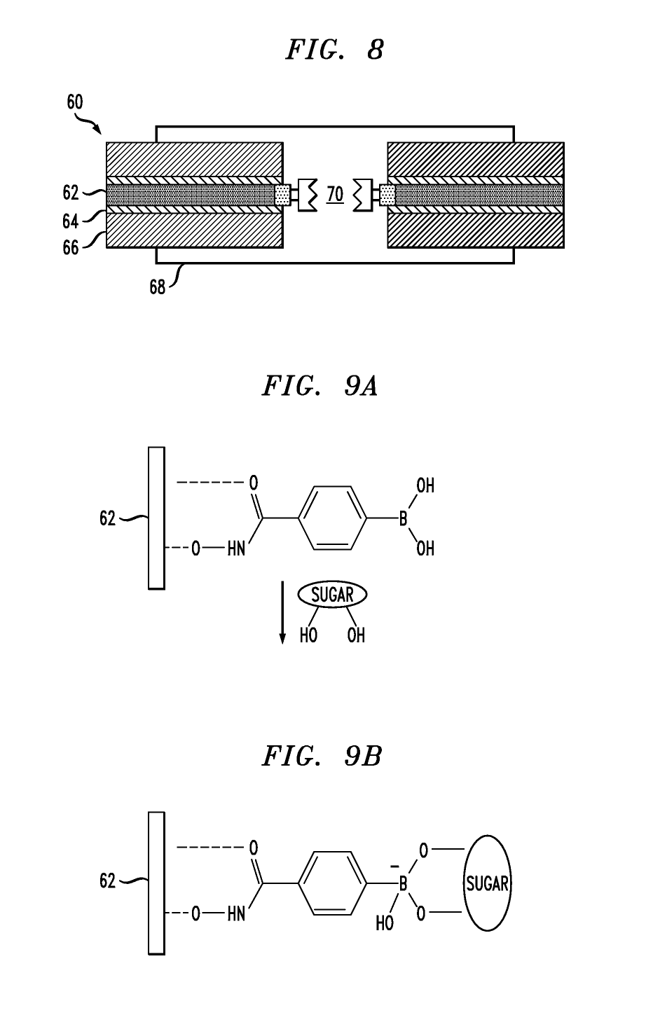

A test device 60 as shown in FIG. 8 can be used to demonstrate the feasibility of the methods disclosed herein. The device includes a twenty (20) nanometer thin film 62 of TiN in a stack comprising dielectric layers 64, 66 of SiO₂ and Si₃N₄ that are 10 nm and 50 nm in thickness, respectively. The device includes a fluidic cell 68 containing a KCl solution. The TiN layer includes a pore 70 less than one hundred nanometers in diameter and preferably smaller. The TiN membrane layer has a plasma oxidized surface in this exemplary embodiment. A receptor layer 72 is bound to the membrane layer 62, reducing the pore size. The Si₃N₄ and SiO₂ dielectric films of the device 60 can be deposited using plasma enhanced chemical vapor deposition (PECVD). The TiN film can be deposited using reactive sputtering. The films can be sequentially deposited on a silicon wafer. In order to make a thin membrane layer 62 through which a pore (or channel in alternative embodiments) can be made, standard lithography can be used to pattern the back side of the wafer such that a via can be etched through the entire silicon wafer using an anisotropic silicon etchant such as KOH or tetramethylammonium hydroxide (TMAH).

Pores can be fabricated using a transmission electron beam microscope (TEM) as small as one nanometer. Other techniques can be employed to provide somewhat larger pores such as electron beam lithography and reactive ion etching. It will be appreciated that channels running parallel to a wafer surface rather than through a membrane can be used in accordance with the principles of the invention. Trench-like channels are likely more amenable to scalable techniques such as photolithography and conventional wet and dry (reactive ion) etching techniques. Channels are also preferred for “lab-on-a-chip” applications as discussed hereafter.

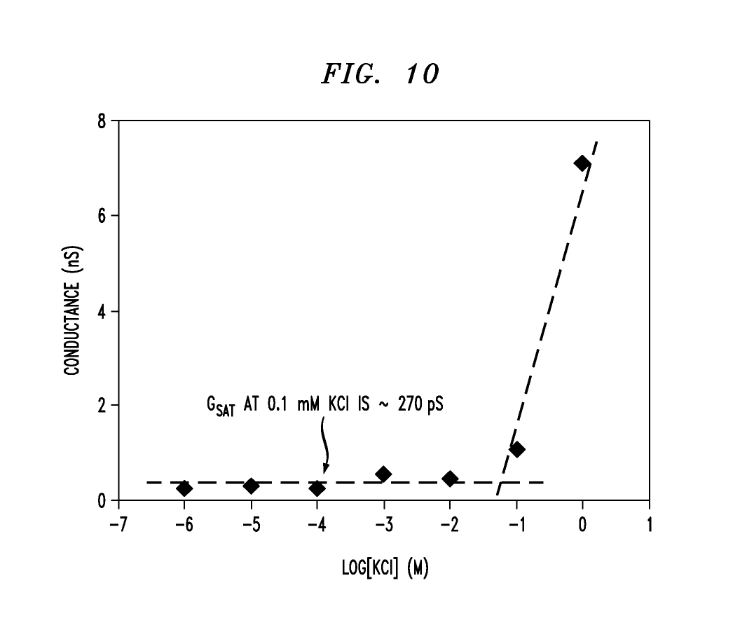

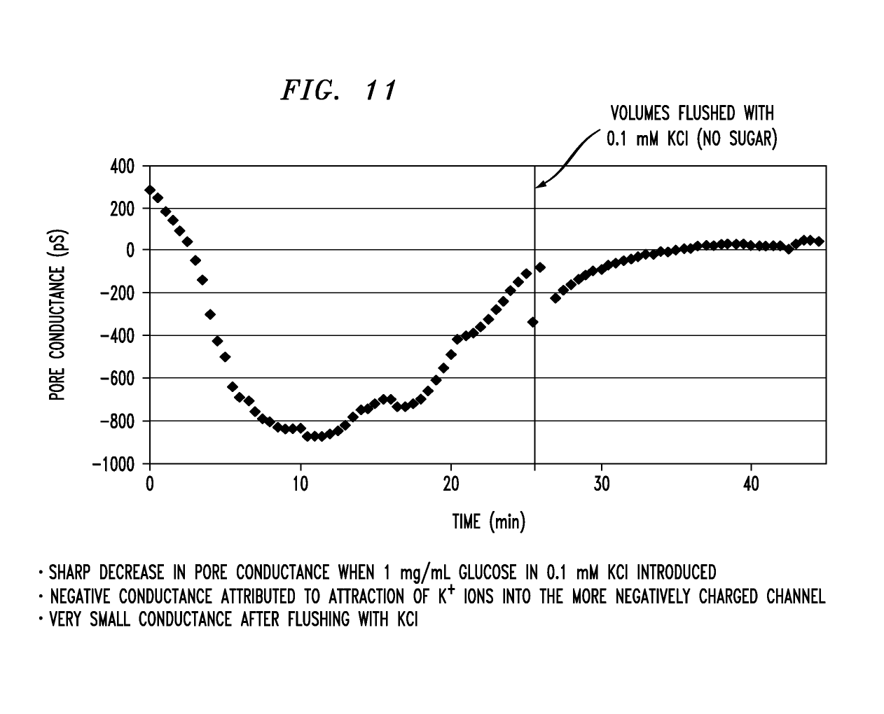

FIGS. 9A and 9B show, respectively, the binding of a boronic acid receptor layer to the surface of the TiN membrane layer 62 and the capture of a D-glucose molecule by the receptor layer. As shown in FIG. 9B, the binding of glucose leads to a negative charge on the boron atom. It will be appreciated that, in addition to glucose, boronic acid can be employed to capture vicinal diols and dihydroxides. It will further be appreciated that pore materials other than TiN can be employed, including but not necessarily limited to TiO₂, Si₃N₄, HfO₂, and Al₂O₃.

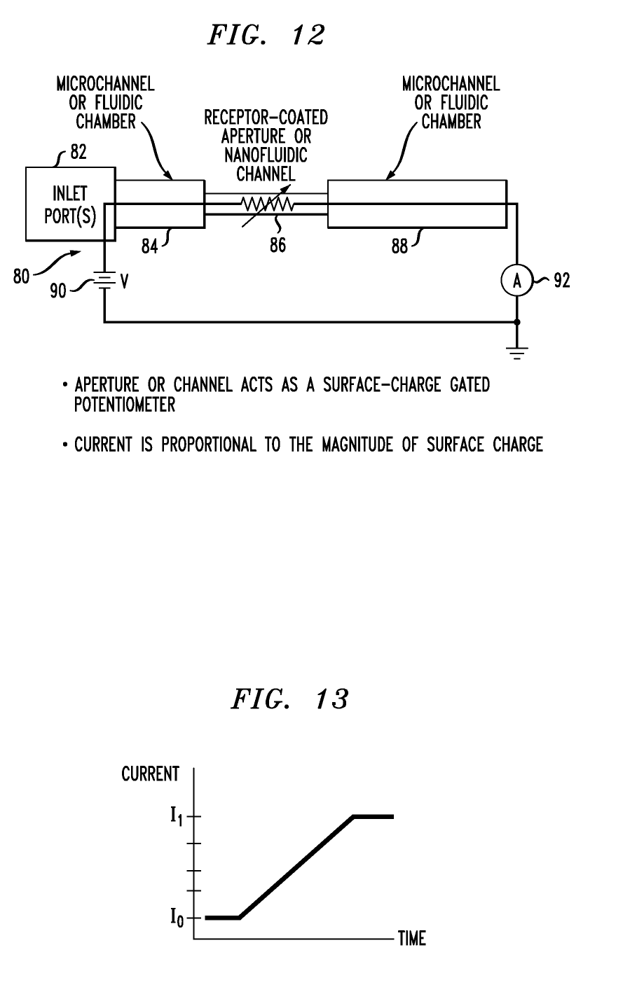

FIG. 10 is a graph showing how the ionic conductance of a device 60 as shown in FIG. 8 changes with salt concentration. In this exemplary embodiment, an 11 nm by 14 nm elliptical pore in the TiN membrane layer 62 is reduced to about 8 nm by 11 nm following oxidation. The boronic acid coating further reduces the pore size to about 6 nm by 9 nm. The data in FIG. 10 shows that the conductance is saturated at a constant value for concentrations of ten (10) mM and below, where this saturated region is the desired region for application of the invention. Following monolayer sugar capture, the pore size would be about 5 nm by 8 nm. 1 mg/mL D-glucose in an 0.1 mM KCl electrolyte solution is provided in the fluidic cell above and below the nanopore. A 100 mV bias across the nanopore is provided. FIG. 11 shows pore conductance in real time following 1 mg/mL glucose introduction into the 0.1 mM KCl electrolyte solution and subsequent flushing of the nanopore with a 0.1 mM KCl electrolyte solution. As shown in the graph, there is a sharp decrease in pore conductance when the glucose-containing electrolyte solution is introduced. The negative conductance is attributed to the attraction of K+ ions in solution to the more negatively charged pore surface. The conductance is very small but non-zero following flushing with the electrolyte solution The complex behavior of the conductance over time can be attributed to transient effects of ionic and molecular diffusion, settling the ionic conductance back at a constant value that is significantly different from the baseline, as glucose will still be bound to the boronic acid within the pore.

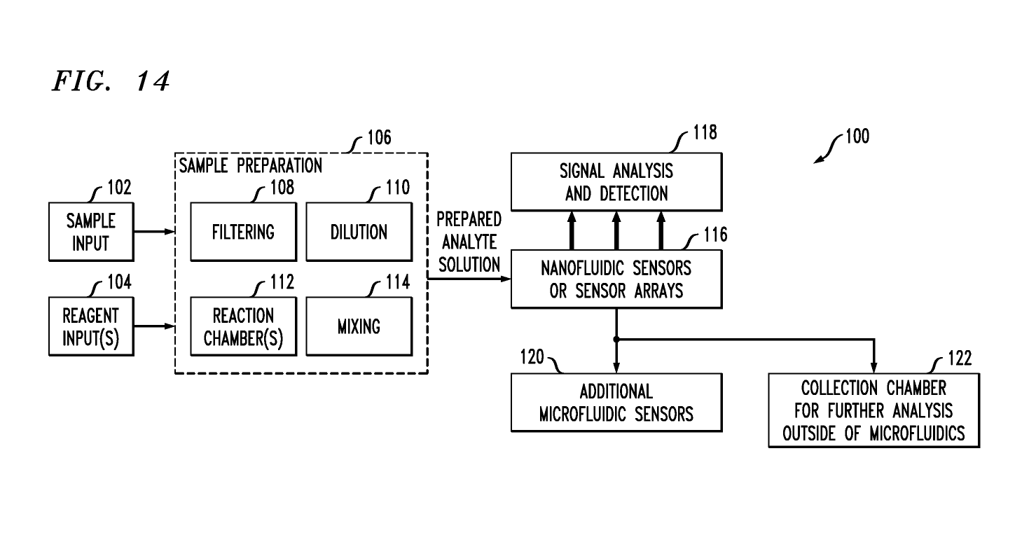

A system 80 for sensing analytes using the principles of the invention is shown in FIG. 12. Such a system may include either pores or channels containing an electrolyte having a sufficiently low salt concentration such that the surface charge (if any) on the pores or channels significantly affects conductance. The system includes one or more inlet ports 82. A fluidic chamber 84, which may comprise a microchannel, is in fluid communication with the inlet port(s). The fluidic chamber 84 can be used, for example, for mixing pure electrolyte solution introduced through a first inlet port with a solution containing (or possibly containing) analyte molecules introduced through a second inlet port. A receptor-coated passage 86 such as a pore or channel is in fluid communication with the fluidic chamber 84 and functions as a sensor. The passage 86, including the receptor layer, has at least one dimension that is one thousand nanometers or less. This at least one dimension is likely to be considerably less than one thousand nanometers for most applications, and would be preferably less than fifty (50) nanometers for many applications. Other dimensions of the passage can be greater than this at least one dimension, possibly substantially greater. For example, a channel could have a depth of one hundred nanometers or less, a width greater than one thousand nanometers, and a length of a micrometer. The relatively large width of the channel can improve the signal to noise ratio. All dimensions of the passage are preferably substantially larger than the maximum dimension of the analyte to be detected, and can be more than ten times larger than the maximum dimension of the analyte. A second microchannel or fluidic chamber 88 is in fluid communication with the passage outlet. A voltage source 90 is provided for applying an electric potential across the passage 86. The current through the system 80 is detected by an ammeter 92. As illustrated in FIG. 12, the receptor-coated passage 86 functions generally like a variable resistor. Changes in current are related to changes in sensor resistance which correlates to changes in passage surface charge. The current is proportional to the magnitude of the surface charge in the passage 86. FIG. 13 shows measured current in the system 80 as a function of time. In this exemplary embodiment, surface charge increases over time.

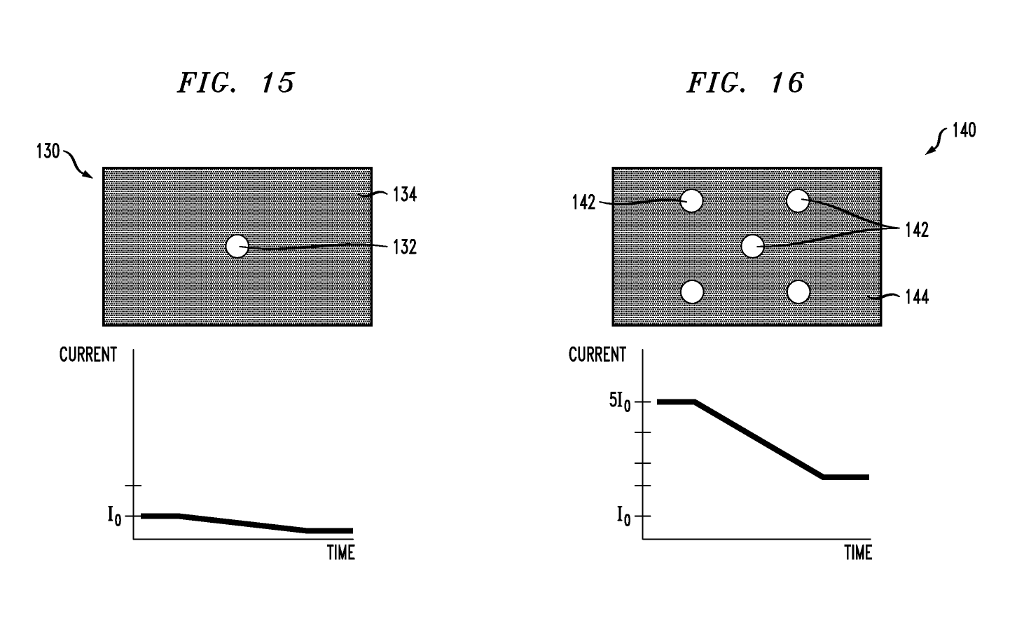

FIG. 14 includes a flow diagram showing the operation of a microfluidic system 100 that can be used for implementing principles of the invention. Various functions performed by the system may be controlled by a computer. Sensors as described herein can be incorporated in microfluidic systems as shown herein or other such systems that may now be available or that become available in the future. The exemplary system includes one or more fluidic inputs that may be used for different samples or, as shown in FIG. 14, a sample input 102 and a reagent input 104. A sample preparation area 106 as shown includes a filtering device 108, a dilution chamber 110, a reaction chamber 112 and a mixing chamber 114. It will be appreciated that the sample preparation area may include more than one of these elements and/or additional elements. The prepared analyte-containing electrolyte solution is fed to a sensor 116 from the sample preparation area 106. The sensor may be, for example, a single nanofluidic passage (pore or channel) having a receptor layer for capturing a targeted analyte or an array of such passages. In accordance with the invention, at least one of the passages includes a receptor layer and the electrolyte solution is sufficiently dilute that passage surface charge strongly affects the electrical conductivity through the passage. If an array of passages is employed, each passage may have the same dimensions or one or more passages may have different dimensions. As discussed above, each passage wherein surface charge is intended to materially affect electrical conductivity therethrough has at least one dimension that is one thousand nanometers or less, preferably fifty nanometers or less for many potential applications. An electrical parameter relating to passage conductivity is obtained using a signal analysis and detection device 118. Such a device may include an ammeter. Analysis of the electrical parameter may provide information relating to the presence of the targeted electrolyte, its concentration, or other information. The system 100 may further include additional microfluidic or nanofluidic sensors 120 that may work in the same manner as the sensor 116 or a different manner. A collection chamber 122 may additionally be provided for receiving prepared analyte-containing solution for further analysis outside the microfluidic system 100. The collection chamber 122 may be in fluid communication with the sensor 116 as shown or with the sample preparation area 106. Opportunities for multiplexed sensing as well as using a variety of sensing technologies can be provided by the system 100.

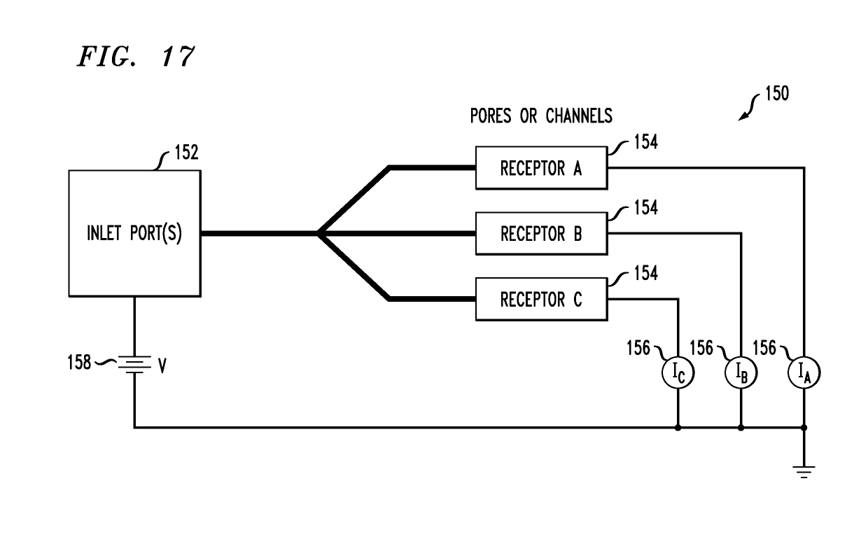

FIG. 15 shows one type of sensor 130 that can be employed as the sensor 116 in FIG. 14. This sensor includes a single pore 132 in a membrane 134 that includes a receptor layer for targeting an analyte that passes through the pore in an electrolyte solution having a very low salt concentration. Current as a function of time for such a sensor 130 is also shown in the figure. The capture of analyte in this exemplary embodiment causes a decrease in the current through the pore. It is further assumed that analyte binding decreases the current by fifty percent, but that noise is about 25% of the original current i₀. It will be appreciated that detecting electrical parameters related to conductance, such as electrical current, is considered the same as detecting ionic conductance for the purposes of the techniques disclosed herein. Electrical current is proportional to the magnitude of the surface charge within a pore 132 or other fluidic passage under the operating parameters disclosed herein.

A second type of sensor 140 that can be employed as the sensor 116 in the system of FIG. 14 is shown in FIG. 16. This sensor 140 includes an array of pores 142 in a membrane 144, each of the pores including a receptor layer for targeting an analyte. The array of pores exhibits a superior signal-to-noise ratio than single-pore sensors, as illustrated by the associated graph.

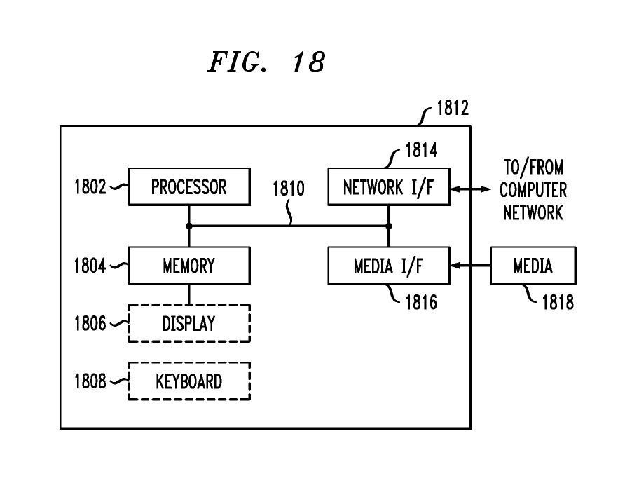

FIG. 17 shows a system 150 similar to the system 80 shown in FIG. 12. The system includes inlet port(s) 152 and passages 154 in fluid communication with the inlet port(s). As discussed above, the passages can be either pores or channels. If channels are employed, at least one dimension of each channel is preferably less than 100 nm. In this exemplary embodiment, each of the passages includes a different receptor layer, designated as Receptor A, Receptor B and Receptor C, respectively. It will be appreciated, however, that the receptor layers can be comprised of the same materials. One passage can function as a control and have no receptor layer. By providing a plurality of receptor layers, the system 150 may be used for multiplexed detection of a plurality of analytes. In this exemplary embodiment, an ammeter 156 is associated with each passage 154. While only one voltage source 158 is shown, it will be appreciated that the system may be configured to include separate voltage sources for each passage. In operation, an electrolyte solution having a low salt concentration travels from the inlet port(s) 152 to the passages 154 and flows simultaneously through the passages. If the electrolyte solution contains different types of analytes, the different receptor layers can be designed to capture the different analytes. Analyte capture is reflected by changes in current that is detected by the ammeters associated with each passage as a result of changes in the surface charges thereof due to analyte binding on the receptor layers. It will be appreciated that the system can include multiplexed arrays of passages, each array having the same receptor layer composition. It will be appreciated that a single ammeter can be employed instead of the plurality of ammeters 156 and arranged to detect electrical current through each passage 154. Current measurements can be obtained sequentially from each passage as analyte-containing electrolyte solution flows through the passages. The measurements can be transmitted to a memory such as memory 1804 in FIG. 18 for storage or further processing. Measurements can be taken continuously or at selected times whether using one or a plurality of ammeters.

As discussed above, pores, channels, and arrays of pores or channels can be used as the fluid passages for practicing the invention. As shown in the figures, pores can have various shapes, including but not limited to the circular and elliptical shapes appearing in FIGS. 1(b)-(d) and 2(b)-(d). Channels formed as trench-like structures in wafer surfaces can also be formed in various configurations. The sizes of the pores or channels are such that surface charges on the walls thereof strongly affect electrical conductance when electrolyte solutions having sufficiently low salt concentrations are present therein. The pore or channel sizes are also dependent in part on the sizes of the analytes that are targeted. With respect to channel-type passages, it is preferable that at least one dimension is less than one hundred nanometers so that surface charges make an effective difference in electrical conduction. Other dimensions of the channel-type passages can be much larger and may preferably be larger for purposes of enhancing the signal to noise ratio. It may be possible to sense some charged analytes flowing through passages having at least one dimension in the microfluidic range (0.1 to 100 microns) as well as the nanofluidic range (1-1000 nm). Passages should be larger than the analytes to be captured therein. For example, antibodies are typically on the order of five to ten nanometers in size. Passages used for capturing such antibodies should have all dimensions larger than five to ten nanometers and preferably in the range of about five to ten times larger than the maximum analyte dimensions. It will be appreciated that analytes may have irregular shapes and that the passages for certain analytes may need to be designed with optimal sizes and shapes to best allow the detection of such analytes through changes in conductance based on changes in surface charge while avoiding pore or channel blockage that would materially impede flow. Passages having at least one or possibly all dimensions at least ten times larger than the maximum dimension of the analyte may be preferred for certain applications.

In addition to the passage size and shape considerations as discussed above, passage length is a further consideration in designing sensors using surface charge techniques. The dynamic range of a sensor depending on passage surface charge changes increases with increasing functionalized pore or channel length. If the receptor layer only constituted a thin portion of the passage, it could easily saturate with analyte molecules because of the limited number of binding sites. By extending the functionalized length of the passage, it takes longer for analyte rich solutions to saturate. Higher concentrations of analytes can accordingly be detected with longer functionalized passages as more binding sites are available. However, for trace detections of materials, maximum sensitivity is desired and dynamic range can be sacrificed.

A secondary “label tag” may be attached to an analyte to provide a charge if necessary. Such a tag may be similar to a receptor molecule, but not tethered to the surface. Secondary tagging techniques are employed in enzyme linked immunosorbent assays (ELISA) using a pair of antibodies “sandwiching” an analyte of interest. The secondary antibody has a tag that can be detected by fluorescence, colorimetry/horseradish peroxidase, radiolabeling, or other techniques. There are, for example, typically many different antibodies for the same protein, but they oftentimes bind to different regions of the protein with different amino acid sequences and/or configurations. The binding in this exemplary embodiment is similar to the capturing of analyte described above, only the binding is to a different part of the analyte. The secondary tag should carry a charge, either naturally or by design, which could then provide the surface charge within the fluidic passage. If a secondary tag is used, the baseline measurement would occur after analyte is captured, i.e. following introduction of the low concentration electrolyte solution that possibly contains the analyte, and the final measurement would occur after this secondary tag/receptor is introduced or bound. The secondary tag/label is specific enough to the analyte that it would only bind to the passage surface if the analyte were present.

A number of different materials can be chosen for use as pore or channel materials, including but not limited to SiO₂, TiN, and Si₃N₄. Au is a further possibility and has been used with thiol-terminated single stranded DNA molecules used as receptors. The surface chemistry of the channels or pores is adaptable for a large number of different molecules in order to tether a particular molecular or enzymatic receptor on the surface.

Transient and steady-state changes in current may be used to provide information relating to an analyte. Steady-state changes would be observed by taking an initial baseline reading of an electrical parameter, introducing the electrolyte solution containing the analyte, and taking an equilibrium measurement at a later time. If the electrical parameter, such as electrical conductance, were measured in real time, additional information relating to the kinetics of the interactions in the passage can be obtained, such as the rate of change of the electrical conductance when exposed to the analyte-containing solution. Transient responses may potentially be affected by diffusion of the analyte, which is a function of concentration and passage size, and the kinetics of binding of the analyte to the receptor layer. For example, an analyte might bind permanently to a receptor molecule or it might tend to disassociate from the receptor layer after initial binding.

The systems and methods provided by the invention take advantage of changes in surface charge of a pore or channel at low ion concentrations that strongly affect electrical conductivity. This allows the capability of detecting even single ions in solution such as heavy metal ions with fluidic devices that are much larger than single atomic ions. In contrast, in systems employing high salt concentrations, the ions in solution quickly screen out the surface charges on the passage walls so that only the bulk, resistor-like salt concentration affects the electrical current. The resistance at such concentrations scales as length over a cross sectional area. Systems relying on high salt concentrations tend to rely on changes in pore cross section due to the binding of analytes that affect current. The techniques employed in accordance with the present invention are fairly insensitive to analyte size as substantial shrinkage of pore or channel dimensions is not a requirement for analyte detection. The ability to detect relatively small analytes with smaller receptor layers is an advantage of the present invention. Very large molecules that are likely to fluctuate and cause significant channel blockage and thereby compete with surface-charge-based signals and appear as added noise to the system may not, however, be ideal candidates for detection using the techniques provided herein.

Given the discussion thus far, it will be appreciated that, in general terms, an exemplary method, according to an aspect of the invention, includes the step of obtaining a device comprising a fluidic passage including a receptor layer for capturing a selected analyte, the fluidic passage including the receptor layer having at least one dimension of one thousand nanometers or less. FIGS. 8 and 14 are illustrative of such a fluidic passage comprising a receptor layer. The method further includes flowing an electrolyte solution containing one or more molecules of the selected analyte through the fluidic passage such that the selected analyte is captured by the receptor layer, the capture of the analyte causing a change in surface charge on the receptor layer. FIG. 7, for example, shows the capture of analyte molecules by a receptor layer while FIGS. 9A and 9B show the capturing step with respect to a specific analyte (glucose) and receptor layer (boronic acid). As discussed above, the electrolyte solution has a sufficiently low salt concentration that surface charge causes a material effect on ionic conductance through the fluidic passage. The method further includes detecting the ionic conductance through the fluidic passage. FIGS. 11 and 13 include graphs showing the detection of ionic conductance, the first graph showing units of conductance (pS) as a function of time and the second graph showing current as a function of time.

It will further be appreciated that an exemplary system according to the invention includes a substrate comprising a fluidic passage having a surface including a receptor layer for capturing an analyte and causing a change in surface charge upon capturing the analyte. FIGS. 12, 14 and 17 show exemplary systems of this type while FIGS. 8 and 9A show receptor layers that can be used in the systems. The fluidic passage including the receptor layer has at least one dimension of one thousand nanometers or less. A first fluidic chamber is in fluid communication with the fluidic passage, as best shown in FIG. 12. A second fluidic chamber is also in fluid communication with the fluidic passage, as designated by numeral 88 in FIG. 12. A voltage source is provided for applying a voltage across the fluidic passage as shown in FIGS. 12 and 17. A detecting device for detects changes in electrical conductance through the fluidic passage. FIG. 12 shows an ammeter 92 and FIG. 17 shows an array of ammeters 156, all of which are responsive to changes in ionic conductance. An electrolyte solution in the first fluidic chamber has a sufficiently low salt concentration that a change in the surface charge resulting from capture of the analyte by the receptor layer causes a material effect in ionic conductance through the fluidic passage when the electrolyte solution is present therein. Accordingly, if analyte is present within the electrolyte solution, it will be captured by the receptor layer, resulting in a change in the surface charge characteristics of the fluidic passage and ionic conductance. If no analyte is present, the surface charge (if any) within the fluidic passage will remain unchanged. The detecting device can be used to detect the presence or absence of changes in ionic conductance due to surface charge changes within the fluidic passage by measuring electrical parameters such as current.

The invention further encompasses testing processes to determine whether or not an analyte is present in a low concentration electrolyte solution. One such process involves the use of a secondary tag or label as described above. Specifically, a first method that does not require a secondary tag comprises flowing an electrolyte solution through a fluidic passage including a receptor layer for capturing a selected analyte and causing a change in surface charge within the fluidic passage upon capturing the selected analyte, the fluidic passage including the receptor layer having at least one dimension of one thousand nanometers or less, the electrolyte solution having a sufficiently low salt concentration that surface charge within the fluidic passage causes a material effect on ionic conductance through the fluidic passage, and detecting the ionic conductance through the fluidic passage. As discussed above, the ionic conductance is materially affected by changes in surface charge within the fluidic passage and is therefore indicative of the presence or absence of the analyte.

If analyte capture is not sufficient to cause a change in surface charge in the fluidic passage, the method can still be used for detecting the analyte through the use of a secondary tag that, when bound to the analyte, provides change in passage surface charge that may be detected. Such a method comprises flowing an electrolyte solution that may or may not contain a targeted analyte through a fluidic passage including a receptor layer for capturing the selected (targeted) analyte, the fluidic passage including the receptor layer having at least one dimension of one thousand nanometers or less. The electrolyte solution has a sufficiently low salt concentration that surface charge within the fluidic passage can cause a material effect on ionic conductance through the fluidic passage. The method further comprises introducing a secondary tag capable of binding with the selected analyte into the fluidic passage and providing a surface charge within the fluidic passage upon binding with the selected analyte. The ionic conductance through the fluidic passage is detected. If analyte is present, the secondary tag will bind to the analyte within the fluidic passage and affect the ionic conductance by the resultant change in surface charge therein.

### Exemplary System and Article of Manufacture Details

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

One or more embodiments of the invention, or elements thereof, can be implemented in the form of an apparatus including a memory and at least one processor that is coupled to the memory and operative to perform exemplary method steps such as measuring ionic current, creating the electric potential across the receptor-layered passage, controlling the flows of electrolyte solution and test sample (possible analyte-containing) solution through the passage, controlling the mixing of electrolyte solution and potential analyte-containing sample, displaying electrical parameters of interest, and storing data relating to the electrical conductivity within the passage. Multiplexed detection of a plurality of materials using arrays on the same chip can be facilitated using a processor and memory. Manufacturing steps for making systems capable of performing the techniques disclosed herein can also be controlled through such an apparatus

One or more embodiments can make use of software running on a general purpose computer or workstation. With reference to FIG. 18, such an implementation might employ, for example, a processor 1802, a memory 1804, and an input/output interface formed, for example, by a display 1806 and a keyboard 1808. The term “processor” as used herein is intended to include any processing device, such as, for example, one that includes a CPU (central processing unit) and/or other forms of processing circuitry. Further, the term “processor” may refer to more than one individual processor. The term “memory” is intended to include memory associated with a processor or CPU, such as, for example, RAM (random access memory), ROM (read only memory), a fixed memory device (for example, hard drive), a removable memory device (for example, diskette), a flash memory and the like. In addition, the phrase “input/output interface” as used herein, is intended to include, for example, one or more mechanisms for inputting data to the processing unit (for example, mouse), and one or more mechanisms for providing results associated with the processing unit (for example, printer). The processor 1802, memory 1804, and input/output interface such as display 1806 and keyboard 1808 can be interconnected, for example, via bus 1810 as part of a data processing unit 1812. Suitable interconnections, for example via bus 1810, can also be provided to a network interface 1814, such as a network card, which can be provided to interface with a computer network, and to a media interface 1816, such as a diskette or CD-ROM drive, which can be provided to interface with media 1818. Interfaces can be provided to microammeters, valves (not shown) controlling electrolyte solution and sample mixing or flow, and/or current supplies and the like over a network or other suitable interface, analog-to-digital converter, or the like.

Accordingly, computer software including instructions or code for performing the methodologies of the invention, as described herein, may be stored in one or more of the associated memory devices (for example, ROM, fixed or removable memory) and, when ready to be utilized, loaded in part or in whole (for example, into RAM) and implemented by a CPU. Such software could include, but is not limited to, firmware, resident software, microcode, and the like.

A data processing system suitable for storing and/or executing program code will include at least one processor 1802 coupled directly or indirectly to memory elements 1804 through a system bus 1810. The memory elements can include local memory employed during actual implementation of the program code, bulk storage, and cache memories which provide temporary storage of at least some program code in order to reduce the number of times code must be retrieved from bulk storage during implementation.

Input/output or I/O devices (including but not limited to keyboards 1808, displays 1806, pointing devices, and the like) can be coupled to the system either directly (such as via bus 1810) or through intervening I/O controllers (omitted for clarity).

Network adapters such as network interface 1814 may also be coupled to the system to enable the data processing system to become coupled to other data processing systems or remote printers or storage devices through intervening private or public networks. Moderns, cable modem and Ethernet cards are just a few of the currently available types of network adapters.

As used herein, including the claims, a “server” includes a physical data processing system (for example, system 1812 as shown in FIG. 18) running a server program. It will be understood that such a physical server may or may not include a display and keyboard.

As noted, aspects of the present invention may take the form of a computer program product embodied in one or more computer readable medium(s) having computer readable program code embodied thereon. Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. Media block 1818 is a non-limiting example. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, such as provided in FIG. 14, and combinations of blocks in the flowchart illustrations and/or block diagrams, can be implemented or facilitate by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be rioted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

It should be noted that any of the methods described herein can include an additional step of providing a system comprising distinct software modules embodied on a computer readable storage medium; the modules can include, for example, any or all of the elements depicted in the block diagrams and/or described herein; by way of example and not limitation, an initialization module, a module to cycle through sample testing, an output module to generate an output file, and a post-processing module providing signal analysis relating to the test samples. The method steps can then be carried out using the distinct software modules and/or sub-modules of the system, as described above, executing on one or more hardware processors 1802. Further, a computer program product can include a computer-readable storage medium with code adapted to be implemented to carry out one or more method steps described herein, including the provision of the system with the distinct software modules. In any case, it should be understood that the components illustrated herein may be implemented in various forms of hardware, software, or combinations thereof; for example, application specific integrated circuit(s) (ASICS), functional circuitry, one or more appropriately programmed general purpose digital computers with associated memory, and the like. Given the teachings of the invention provided herein, one of ordinary skill in the related art will be able to contemplate other implementations of the components of the invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A method comprising:
obtaining a device comprising a fluidic passage including a receptor layer for capturing a selected analyte, the fluidic passage including the receptor layer having at least one dimension of one thousand nanometers or less;
flowing an electrolyte solution containing one or more molecules of the selected analyte through the fluidic passage such that the selected analyte is captured by the receptor layer, the capture of the analyte causing a change in surface charge on the receptor layer, the electrolyte solution having a sufficiently low salt concentration that surface charge causes a material effect on ionic conductance through the fluidic passage, and
detecting the ionic conductance through the fluidic passage.

2. The method of claim 1, wherein at least one dimension of the fluidic passage is greater than one thousand nanometers.

3. The method of claim 1, wherein the fluidic passage has at least one dimension of fifty nanometers or less.

4. The method of claim 1, wherein the receptor layer comprises boronic acid and the analyte is a vicinal dihydroxide.

5. The method of claim 1, wherein the device includes a plurality of fluidic passages, each having a receptor layer for capturing at least one selected material and at least one dimension of one thousand nanometers or less, further comprising flowing the electrolyte solution simultaneously through the plurality of fluidic passages and detecting the ionic conductance through each of the fluidic passages.

6. The method of claim 5, wherein the receptor layer of each fluidic passage is comprised of the same material for capturing the selected analyte.

7. The method of claim 5, wherein the receptor layers for at least two of the fluidic passages are comprised of different materials for capturing different selected analytes.

8. The method of claim 1, wherein the at least one dimension of the fluidic passage is between five to ten times the maximum dimension of the analyte in the electrolyte solution.

9. The method of claim 1, further comprising comparing the detected ionic conductance with a reference.

10. The method of claim 1, wherein the receptor layer comprises single stranded DNA and the analyte is a molecule including a complementary sequence to the single stranded DNA in the receptor layer.

11. The method of claim 1, wherein the receptor layer comprises an antibody and the analyte is a molecule containing an epitope recognized by the antibody.

12. The method of claim 1, wherein the receptor layer comprises an enzyme and the analyte is a molecule acted upon by the enzyme.

13. The method of claim 1, further comprising flowing analyte-free electrolyte solution through the fluidic channel, detecting the ionic conductance through the fluidic passage while the analyte-free electrolyte solution is present in the fluidic passage, and comparing the detected ionic conductance of the analyte-free electrolyte solution with the detected ionic conductance of the electrolyte solution containing the selected analyte.

14. A system comprising:
a substrate comprising a fluidic passage having a surface including a receptor layer for capturing an analyte and causing a change in surface charge upon capturing the analyte, the fluidic passage including the receptor layer having at least one dimension of one thousand nanometers or less;
a first fluidic chamber in fluid communication with the fluidic passage;
a second fluidic chamber in fluid communication with the fluidic passage;
a voltage source for applying a voltage across the fluidic passage;
a detecting device for detecting changes in ionic conductance through the fluidic passage, and
an electrolyte solution in the first fluidic chamber having a sufficiently low salt concentration that a change in the surface charge in the fluidic passage resulting from capture of the analyte by the receptor layer causes a material effect in ionic conductance through the fluidic passage when the electrolyte solution is within the fluidic passage.

15. The system of claim 14, wherein the fluidic passage including the receptor layer has at least one dimension of greater than one thousand nanometers.

16. The system of claim 14, wherein the fluidic passage including the receptor layer is a channel having at least one dimension of one hundred nanometers or less.

17. The system of claim 14, wherein the fluidic passage including the receptor layer has at least one dimension of fifty nanometers or less.

18. The system of claim 14, wherein the substrate further comprises a plurality of fluidic passages in fluid communication with the first and second fluidic chambers, each fluidic passage including a receptor layer for capturing a selected material.

19. The system of claim 18, wherein the receptor layer of each fluidic passage is comprised of the same material for capturing the same analyte.

20. The system of claim 18, wherein one or more of the fluidic passages includes a receptor layer comprised of a material that is different from at least one of the other fluidic passages.

21. The system of claim 14, wherein the dimensions of the fluidic passage are all at least ten times the maximum dimension of the analyte.

22. A method comprising:
flowing an electrolyte solution through a fluidic passage including a receptor layer for capturing a selected analyte and causing a change in surface charge within the fluidic passage upon capturing the selected analyte, the fluidic passage including the receptor layer having at least one dimension of one thousand nanometers or less, the electrolyte solution having a sufficiently low salt concentration that surface charge within the fluidic passage can cause a material effect on ionic conductance through the fluidic passage, and
detecting the ionic conductance through the fluidic passage.

23. The method of claim 22, wherein the fluidic passage including the receptor layer has at least one dimension of fifty nanometers or less.

24. A method comprising:
flowing an electrolyte solution through a fluidic passage including a receptor layer for capturing a selected analyte, the fluidic passage including the receptor layer having at least one dimension of one thousand nanometers or less, the electrolyte solution having a sufficiently low salt concentration that surface charge within the fluidic passage can cause a material effect on ionic conductance through the fluidic passage;
introducing a secondary tag capable of binding with the selected analyte into the fluidic passage and providing a surface charge within the fluidic passage upon binding with the selected analyte, and
detecting the ionic conductance through the fluidic passage.

25. The method of claim 24, wherein the selected analyte is present within the electrolyte solution, further comprising obtaining a baseline measurement of ionic conductance following capture of the analyte by the receptor layer and obtaining a further measurement of ionic conductance following binding of the secondary tag with the analyte.
